(19) United States
(12) Patent Application Publication
(10) Pub. No.: US 2005/0054935 A1
(43) Pub. Date: Mar. 10, 2005

(54) Hyper-spectral means and method for detection of stress and emotion

(76) Inventors: Robert R. Rice, Simi Valley, CA (US); David A. Whelan, Newport Coast, CA (US)

Correspondence Address: HARNESS, DICKEY & PIERCE, P.L.C., P.O. BOX 828, BLOOMFIELD HILLS, MI, 48303, US

(21) Appl. No.: 10/657,338

(22) Filed: Sep. 8, 2003

(51) Int. Cl. A61B 5/05; A61B 6/00

(52) U.S. Cl. 600/473; 600/476; 348/77

(57) ABSTRACT

A system and method for detecting physiological stress in a person is provided which includes an imagining device and a processor. The processor receives an output from the imagining device representative of the. The processor identifies a characteristic of the person in the image which indicates that the person is not experiencing stress and a second characteristic which indicates the person is experiencing stress. A comparator compares the image of the subject and the two characteristics and the processor extrapolates from the comparison whether or not the person is experiencing a heightened state of stress or nervousness. If so the processor provides an output to activate an alarm or otherwise signal this condition to other individuals involved in monitoring a given area.

### FIELD OF THE INVENTION

The present invention relates to the detection of stress in human beings, and more particularly to using hyperspectral methods to detect physiological stress in human beings.

### BACKGROUND OF THE INVENTION

Sep. 11, 2001 vastly increased the need to unobtrusively detect stress in human beings, particularly in individuals about to commit an atrocity. If the individuals responsible for the destruction that occurred on Sep. 11, 2001 could have been detected based on observable signs of stress without the individuals noticing the surveillance, events of that day may have been far different.

More mundane needs to detect human stress also exist. For instance, heavy equipment operators who are becoming fatigued experience stress before they subjectively notice their fatigue. Likewise pilots, truck drivers, air traffic controllers, and mass transit and public transportation drivers are similarly situated. Generally, any worker who might endanger others may become tired and therefore pose a hazard to others and also to property. Automatic, objective means to detect the stress associated with their fatigue could save innumerable lives and untold sums otherwise expended in repairing and replacing damaged property.

In particular, unobtrusive means to detect stress would also be highly desirable. In the related applications of stress detection in anti terror and law enforcement efforts, knowledge of the stress detection system would likely cause the subject to alter his/her behavior to avoid stress detection and subsequent identification as a target or suspect. In fatigue detection applications, typical subjects might find the presence of monitoring equipment offensive or insulting. Moreover, in all of these situations innocent third parties possess civil rights which shield them from intrusive violations of their privacy. Thus, a long felt need exists to unobtrusively detect stress.

Human subjects react to transient physiological stress in a variety of ways including increased pulse rate, muscle tremor, perspiration, and sub-dermal blow. By monitoring the subject for these stress symptoms the presence of the stress may be detected. Polygraph machines monitor pulse, respiration, and galvanic skin response while the subject is interrogated. His/her responses as measured allow, to an extent, an observer to evaluate the truthfulness of his/her responses to an extent. Unfortunately, polygraph machines remain difficult to use, stressful for the subjects, require a highly trained operator, and are difficult to miniaturize sufficiently to become portable. The fatal flaw possessed by polygraph machines, though, lies in their untrustworthiness.

In the alternative, fMRI (functional magnetic resonance imaging) machines have been used to detect stress. However fMRI machines also remain large and expensive. These disadvantages prohibit use of fMRI machines to detect stress in many situations including detecting terrorists at airports and other locations, interrogation of witnesses, and many other applications.

Hyper-spectral image processing has been used for long range unobtrusive reconnaissance but not for detecting stress. Hyper-spectral imaging involves the monitoring of a scene of interest at one or more selected wavelengths of electromagnetic radiation. The selected wavelengths are chosen because the scene is likely to contain a subject of interest which is clearly visible at those wavelengths. Clarity may occur because of the intensity of the particular subject, or because of the contrast of the subject with the background, at the selected wavelength. Additionally, the wavelength selected may be chosen because the background is unlikely to contain other objects which emit or reflect radiation at that wavelength. In the alternative, it may be that the subject is camouflaged, usually imperfectly. If the imperfections allow radiation of a particular wavelength to escape, then that wavelength can be advantageously monitored.

Because hyper-spectral image processors only monitor select wavelengths, the processing power required may be greatly reduced over devices monitoring large bands of the entire spectrum. Accordingly, a less powerful (and less expensive) processor may be used. In the alternative, a greater number of targets may be monitored or the monitored scene may be expanded. Moreover, because hyper-spectral imaging may be accomplished with machine vision systems, no human intervention is necessary. Though human participation may be desirable to supplement the unobtrusive hyper-spectral processor.

While observers of a stressed person can readily detect the presence of that stress, a need exists for an apparatus which automatically detects observable symptoms of stress and which triggers an alarm. Additionally, because human sensory perceptions possess limited abilities to discern subtle changes, a need exists for a more sensitive detection system for such stress. Moreover, because observers can err, tire, or be distracted, a need exists for an automated method to accomplish stress detection.

Such unobtrusive stress detection could be advantageously employed in numerous other applications. For instance, law enforcement personnel investigating crimes could benefit from knowing when a witness or target of an investigation is under stress due to attempting to tell a lie. Retail store owners could benefit from detecting suspected shoppers who are experiencing stress due to their attempt to steal merchandise. Even children with behavioral or learning disorders could benefit from early, reliable detection of stress whereby their care givers can intervene early. Thus a long felt need exists to unobtrusively detect stress.

### SUMMARY OF THE INVENTION

The present invention uses techniques from hyper-spectral processing to detect transient changes of sub-dermal blood flow and dermal hydration (i.e. a stress induced blush causing reddened sweaty skin). Hyper-spectral imaging is a technology in which a given scene is viewed generally in a large number of selected wavelengths and the images recorded for later processing. Immediate, real-time processing may also be used. In some wavelength ranges, features of an observed scene will appear which are easily detectable and obvious, whereas these same features might exhibit low contrast and visibility at other wavelengths. Thus, wavelengths are selected for use in hyper-spectral systems according to whether they convey information in which the observer is interested.

Usually the subject will be observed against a complex background that tends to mask the presence of the subject. For instance, the background may reflect or emit a spectrum including a variety of ranges which over lap the selected wavelengths. For instance, marijuana growing in a forest may be masked from law enforcement surveillance by the various shades of green of the forest, unless the surveillance occurs at a “green” wavelength unique to marijuana. More particularly, some targets will employ techniques to mask their presence by using aids to alter their reflected spectrum. An example of such a situation would be the use of camouflage netting to conceal a command post or artillery battery.

An underlying principle of the present invention is that the “color,” or reflectance spectrum, of the skin of a person is modified as a result of transient changes in dermal hydration and sub-dermal hemoglobin flow associated with the emergence of a blush. The blush may be induced by stress or other physiological arousal. In accordance with the present invention, these changes may be passively and unobtrusively detected.

While observers of the blushing person can readily detect the blush once it progresses far enough, a need exists for an apparatus which can automatically detect the emergence of a blush and trigger an alarm. Additionally, because the human eye is limited in its ability to discern subtle changes in coloration (the reflection spectrum) a need exists for automated detection of physiological stress. Moreover, because observers can err, tire, or be distracted, a need exists for a machine to accomplish stress detection.

In addition to satisfying those needs, the present invention accounts for intervening reasons which may alter the reflectance spectrum of a person's skin. A database which characterizes typical skin types (i.e. colors) under controlled conditions and subject to a variety of factors (such as age, sex, cultural background and ethnicity) may be consulted to improve the accuracy of the hyper-spectral processing system. Accordingly, the database enables the identification of a hyper-spectral signature for stress despite the presence of these intervening factors.

The present invention also provides a hyper-spectral system which monitors wavelengths selected based on the considerations discussed herein. Image processing software, decision making software, and display and communication interfaces are also included in accordance with preferred embodiments of the present invention. A spectral instrument, in accordance with a preferred embodiment of the present invention, may be implemented in a portable configuration which operates passively and unobtrusively without physical contact with the subject or his/her awareness of surveillance.

In accordance with a preferred embodiment of the present invention a circuit for detecting physiological stress in a specimen is provided which includes an input and a processor. The input receives an image from a camera and provides it to the processor. The processor identifies two characteristics of a subject who is within the image. The first characteristic indicates that the subject is not experiencing stress and the second characteristic indicates the subject is experiencing stress. A comparator compares the image of the subject and the two characteristics. If the subject appears to be stressed an alarm is signaled.

In accordance with a second preferred embodiment of the present invention a method of detecting physiological stress of a subject is provided. The method includes observing the subject who includes a first spectral characteristic when unstressed and a second spectral characteristic when stressed. The image is compared to the first and the second characteristics to determine whether the subject is stressed.

In accordance with a third preferred embodiment of the present invention a circuit for detecting physiological stress in a subject is provided. The circuit includes a processor which receives an image of the subject. Two areas of the subject's skin are identified by the processor. One area of skin is unlikely to blush and the other area is likely to blush. By comparing the two areas of skin, the processor identifies attenuation of one of the areas of skin which is indicative of a blush.

Further areas of applicability of the present invention will become apparent from the detailed description provided hereinafter. It should be understood that the detailed description and specific examples are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a block diagram of a stress detection system in accordance with a preferred embodiment of the present invention;

FIG. 2 is a graph of typical reflectance spectra from a variety of human skin types;

FIG. 3 is a graph of a typical reflectance spectrum of human skin;

FIG. 4 is a graph of the absorption spectrum of human hemoglobin;

FIG. 5 is a graph of the extinction coefficient of water; and

FIG. 6 is a flowchart of a method in accordance with a preferred embodiment of the present invention.

### DETAILED DESCRIPTION OF THE PREFERRED EMBODIMENTS

The following description of the preferred embodiment(s) is merely exemplary in nature and is in no way intended to limit the invention, its application, or uses.

Normally ambient light reflected from a person's skin determines the spectrum of light which an observer sees and interprets as color. It should be noted that light, herein, refers to more than visible light. For the term light encompasses electromagnetic radiation, in particular visible and near infrared radiation in the ranges of approximately 350 to 700 nanometers and 700 to 1500 nanometers respectively.

Those experiencing physiological stress exhibit a number of conditions observable via such radiation. Colloquially, they tend to blush and perspire. The red, sweaty face of a blushing person belies his/her stress. In more scientific terms a blush is an increase in the sub-dermal hemoglobin (blood) flow. More particularly, the hemoglobin visible seen during a blush is generally oxygenated hemoglobin (i.e. red blood).

Dermal hydration, or an increase in perspiration, also typically accompanies a blush. That perspiration, or sweat, contains mostly water with sodium chloride (disassociated sodium and chlorine ions), potassium, magnesium, skin oils, and other trace chemicals in solution. Thus, during a blush a thin film of water tends to cover the skin.

To understand the present invention it is useful to review how light reflects off of skin. Instead of merely reflecting off of skin, ambient light penetrates the epidermis, the upper layer of skin, and is reflected back to the surface. During a blush, incident light approaches the epidermis through the film of perspiration. The film may be infinitesimally thin to about a tenth of an inch deep (where a drop or rivulet has formed). Because water blocks certain wavelengths of electromagnetic radiation, the film of perspiration alters the spectrum of the incident radiation. Notably the perspiration attenuates the intensity of the radiation at those wavelengths at which it absorbs the radiation.

Once the incident radiation enters the epidermis, the epidermis scatters and absorbs some radiation. The remainder it reflects back toward the surface of the skin. It should be noted that normal ambient light only penetrates the epidermis to a depth of about 0.08 inches. Within that region it begins encountering hemoglobin filled capillaries within about the first 0.001 inches. For neonates, as opposed to adults, the amount of blood content ranged from about 4 to about 12 milligram of hemoglobin per gram of tissue (equivalent to about 0.8 to about 2.4% by volume) and the average depth of blood ranged from about 250 to about 425 micrometers as reported by S. L. Jacques, I. S. Saidi, and F. K. Tittel, Average Depth of Blood Vessels in Skin and Lesions Deduced By Optical Fiber Spectroscopy, Society of Photo-Optical Instrumentation Engineers Proceedings of Laser Surgery: Advanced Characterization. Therapeutics, and Systems IV, edited by R. R. Anderson, 2128, 231-237 (1994).

Because of the capillaries, hemoglobin also absorbs a portion of the incident radiation proportional to the amount of hemoglobin which the radiation encounters. Once reflected out of the epidermis, the reflected radiation again encounters the perspiration which absorbs still more of the incident (now reflected) radiation. Thus, the reflected radiation carries, within its altered spectrum, information indicative of the amount of perspiration and sub-dermal hemoglobin present. In particular, the reflected radiation shows a proportional decrease in intensity at the wavelengths absorbed by water and by hemoglobin. Accordingly, the reflected spectrum indicates the degree to which the person is blushing. Since a blush indicates stress, the reflected spectrum indicates the extent to which the person is under stress.

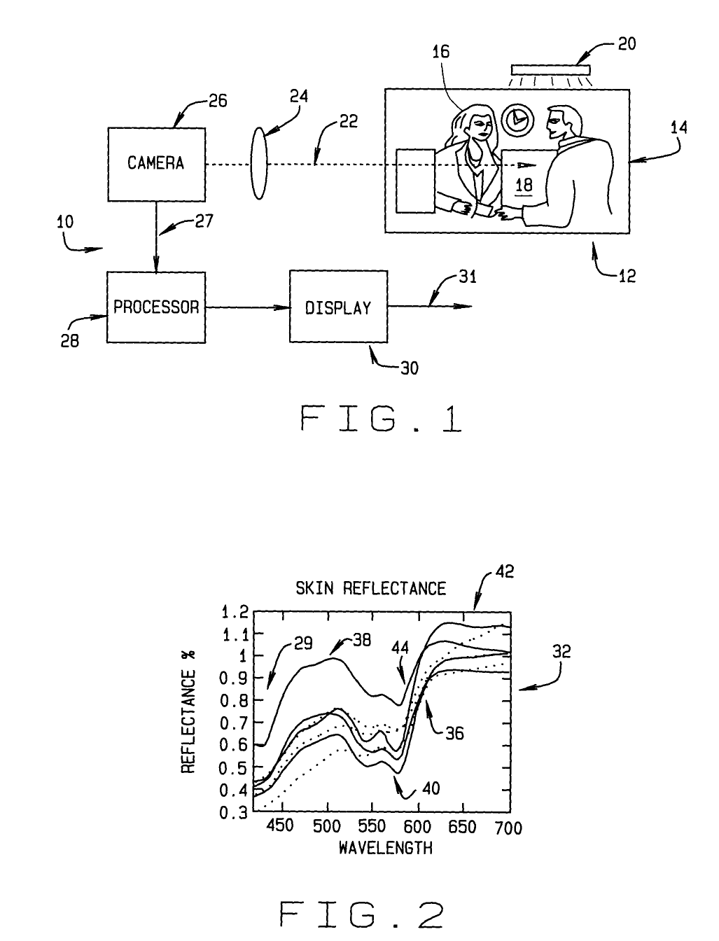

Referring in general to the figures and in particular to FIG. 1, a hyper-spectral system 10 in accordance with a preferred embodiment of the present invention for detecting physiological stress may be seen. The system 10 views, observes, or surveys a scene 12 of interest. Within the scene 12 a subject 14 may be surveyed. Also, as part of a background 18, other non suspect persons 16 may be present as well as vegetation, equipment, and other objects. The subject 14 (or specimen suspect, or target) may be experiencing stress and accordingly may be blushing to some degree. In particular, the subject 14 may be attempting to suppress his/her blush. Yet, because blushing is a partially involuntary reaction to stress, the subject 14 will not be entirely successful in suppressing his/her blush. To aid in the detection of full, and even partial blushes, a data base of numerous varied subjects may be collected which would include data defining their normal, non blushing, skin reflectance spectra and their skin reflectance spectra as altered by the presence of a full blush. Moreover, the data may reflect the subjects as seen in various environments to enable statistical analysis of the spectra in the data base and also that of the spectra captured from images of the subject(s) 14.

To illuminate the scene 12 a visible light source 20 may be included or augmented to illuminate the scene 12. While the light source 20 may be a conventional light source, it may also contain an infrared radiation source. In the alternative, the source 20 could be natural or even diffuse light. Electromagnetic radiation from the source 20, including visible light and preferentially near infrared radiation, illuminates the subject 14. Subject 14 in turn reflects the radiation while altering the spectrum of the incident radiation because of inherent and transient characteristics of his/her skin. More particularly, dermal hydration and oxygenated hemoglobin, indicative of a blush, may attenuate certain wavelengths of radiation in the reflected spectrum.

To receive an image 22 of the scene 12, including the subject 14, the system 10 has a lens or other optical device 24 which focuses the image 22 on a receptor within a hyper-spectral imaging camera 26. Camera 26 may be any type of electronic camera readily available such as a charge coupled device (CCD) or a complementary metal oxide system (CMOS) device capable of sensing either, or both, visible and infrared radiation. The camera 26 captures the image 22 as an array of pixels 27. Note should be made that the camera 26 may operate with ambient, indoor radiation alone. In particular, no laser or other high intensity radiation source need be employed to capture the image 22. Though such high intensity sources, or additional conventional sources, may be employed if it is desired to increase the signal to noise ration of the image, particularly at the selected wavelengths.

From the camera 26, the pixel array 27 is sent to a signal processor 28 as shown in FIG. 1. Note that the camera 26 or the signal processor 28 may filter the image so that only those frequencies of interest may be selectively examined. More particularly, the selected frequencies may be only those at which a blush changes the skin reflectance spectra or just one of these frequencies. Though, other frequencies could be examined also, or ignored, without departing from the spirit or scope of the present invention.

Within the image processor 28 a machine vision application may recognize the shape of human beings. Once the processor 28 recognizes one or more humans it may then prioritize them for further scrutiny. In one embodiment, the system allows a user to select the prioritization scheme. These schemes include prioritization factors such as proximity to the camera 26, proximity to a security station (e.g. checkpoint), similarity to a pre selected photograph, or the presence of indicia of membership in some group (e.g. military insignia), particularly terrorist groups.

Once a target or subject 14 has been selected, the image processor 28 searches for exposed areas of the skin of the subject 14. Such a search may be based upon identifying areas of the image 22 with spectrum similar to those shown in FIG. 2. In the alternative a user with a computer mouse, joystick, light pen or other pointing device may direct the image processor 28 to an area to scrutinize.

Returning now to FIG. 1, once the image processor 28 has identified an area of exposed skin, the image processor 28 may attempt to determine which inherent skin type (i.e. color) the subject 14 possesses. The determination of the skin type may be based on the overall albedo of the subject 14 and a look up table of different skin types according to albedo. In the alternative, the skin type determination may be by way of a hyper-spectral analysis of the reflected ambient light from the subject 14 and comparison to the skin types of, for example, FIG. 2 which may be stored in a database. Either way, the processor 28 obtains a skin reflectance spectrum (such as spectrum 34) against which to compare a spectrum which might exhibit a blush.

The former alternative (albedo based approach) allows for a quicker, less computationally intensive examination while the latter alternative improves the accuracy of the system. In one preferred alternative the albedo based approach executes first to provide a quick assessment. The hyper-spectral analysis approach then executes (or executes in parallel with the albedo based approach) to confirm the results of the quicker approach.

After, or in parallel with, determining the inherent skin type, the image processor 28 then performs a hyper-spectral examination of another area of the skin of the subject 14. With the knowledge of the two partial spectra gained from the examinations the processor compares the two partial spectra searching for difference between the two areas of skin indicative of a blush.

Having completed the comparison, the image processor 28 may then forward the results of the comparison (including the associated images) to a display and alarm device 30 for the user to view or may forward the results to a computer network 31. If the results prove negative (no blush) security personnel may allow the prior subject 14 to pass since his/her classification may now change to that of a non suspect person 16. However, if the results prove positive (the subject 14 is blushing) the image processor 28 may alert security personnel via the display 30 or an alarm.

Moreover, the image 22 may be sent to the network 31 of FIG. 1 for filing and subsequent data processing. In particular, time, date, and location information may be associated with the image 22 to allow subsequent intelligence analysis of the image 22 and subject 14. Thus, the network 31 may connect to intelligence, law enforcement, and other appropriate computers via the internet and other connection schemes.

Since it may be advantageous to allow even a blushing subject 14 to proceed on his/her way (e.g. to expose the remainder of his/her support network or accomplices), the system 10 may be concealed in, or near, the scene 12. In this manner the subject 14 proceeds unaware of the surveillance and his/her potential identification as an individual under stress. Such an unobtrusive surveillance system allows security personnel to check the target's appearance and take further action as may be required. In the alternative, security personnel may choose to allow a stressed person they deem to be innocent to proceed with out complication.

While the present invention has heretofore been described as operating with electromagnetic radiation, the present invention is not so restricted. Any energy which radiates in waves, such as sound, may be utilized to detect signs of stress in the subject in accordance with the present invention. Notably, hyper-spectral analysis of sound may be used to detect an increased pulse of the subject as discussed in U.S. Pat. No. 5,867,257 issued to Rice et al and incorporated herein by reference in its entirety.

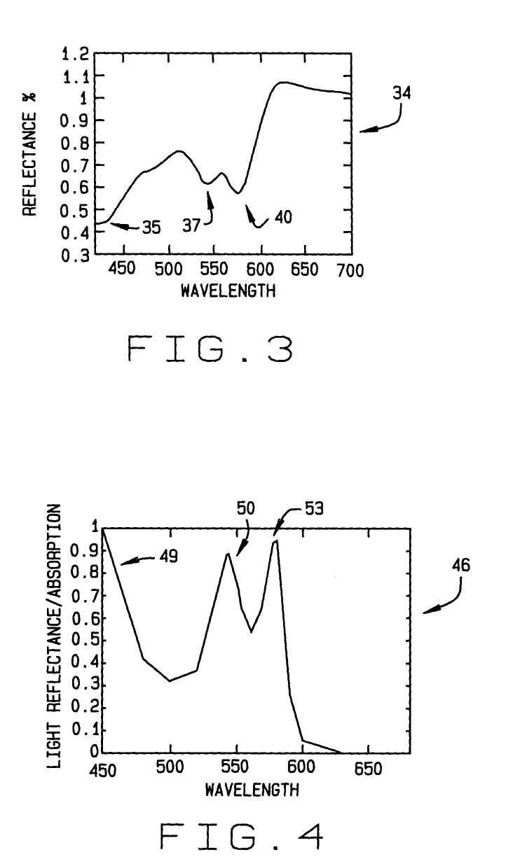

Turning now to the image 22 in more detail, reference is made to FIG. 2. FIG. 2 appeared in Elli Angelopoulou, The Reflectance Spectrum of Human Skin, Technical Report MS-CIS-99-29 (December 1999) (unpublished manuscript on file with the Technical Reports Librarian, Department of Computer and Information Science, University of Pennsylvania, 200 S. 33rd Street, Philadelphia, Pa. 19104-6389). FIG. 2 shows the reflectance spectra for the back of the hand for various types of skin. Because the extremities tend to not participate in blushes the back of the hand is of particular use in the present invention. By a spectral examination of the back of the subject's hand, the image processor 28 may observe, identify, and characterize a typical base line, non blushing, skin reflectance spectrum 34 (FIG. 3) for the particular subject 14.

In particular, because the back of the hand and the face of the subject 14 are likely to be exposed to approximately equal amounts of ultraviolet radiation (e.g. the sun or tanning booths), the amount of melatonin, which dominates which type of skin the subject 14 has, will be approximately equal between the hand and the face. That is to say, the face and the back of the hand will be tanned approximately equally, thereby avoiding one intervening factor, tanning, which may cause the system 10 to detect false positive or negative blushes.

For subjects 14 with skin containing high amounts of melatonin obtaining a real-time non blushing spectrum is of particular importance in suppressing false alarms. That result follows from the tendency of high melatonin skin to have a flatter reflectance spectrum than other skins. Accordingly, these skins attenuate the incident radiation to a greater degree with or without a blush present. As an aside, because people tend to swing their hands slightly as they walk, a machine vision application associated with the image processor 28 may be easily programmed to detect the hand by the swinging motion.

Note should be made of several characteristics of human skin shown in FIG. 2. First the skin reflectance intensity 36 generally tends to increase with increasing wavelength. Though a local maximum 38 tends to occur near 500 nanometers with corresponding local minimums 29 and 40 near 375 and 575 nanometers respectively. A plateau 42 also tends to occur at and above 600 nanometers with a high derivative area 44 connecting the local minimum with the plateau 42. By searching for these features of the image 22 of the subject 14, the image processor 28 may determine areas of skin visible on the subject 14. From these areas, the image processor may then extract at least one base line, skin reflectance spectrum 34 (see FIG. 3). Extracting more than one base line, skin reflectance spectrum 34 may be useful in mitigating the presence of scars, skin grafts, tattoos, port wine stains, and deliberate skin alterations to camouflage the subject 14.

While the features 29, 38, 40, 42, and 44 tend to appear in all skin types shown in FIG. 2, darker skin types exhibit a flatter, less intense spectrum than other skins. Thus, the image processor 28 may contain or access a database of other features of the spectra 32 (shown in FIG. 2) to aid in distinguishing skin from other objects in the image 22 and to enable the selection of a base line reflectance spectrum 34. With regard to the spectrum, it will be understood by those skilled in the art that the mention of specific wavelengths herein will be understood to include a sufficient tolerance to accommodate measurement inaccuracy, variations between skin types, variations between individuals, and variations between different areas of the subject's body, and variations of the subject's skin over time.

As mentioned previously, the processor 28 could measure the overall albedo of the subject 14 and then look up a skin type with a corresponding albedo to improve the speed of the system. However, one albedo may correspond closely with several skin types 32 having different spectrum. To account for such a possibility, an algorithm to choose between the alternatives may be executed by the processor 28. However, a full spectral analysis of a non blushing area of the subject 14 allows the processor 28 to make the blush determination using the subject's current skin type. Thus tanning, skin bleaching, and other attempts to camouflage the subject 14 may be more easily defeated.

It should be noted, prior to discussing the comparison between a blushing and non blushing area of skin, that the typical base line, skin reflectance spectrum 34 (of FIG. 3) may exhibit some hemoglobin caused attenuation. The reason for the attenuation is that even when the subject 14 is nominally unstressed, his/her skin contains some oxygenated hemoglobin. Thus, three local minimums occur on the base line, skin reflectance spectrum 34: minimums 35, 37, and 40. These minimums correspond to the peaks 49, 50, and 53 which appear on the hemoglobin absorption spectrum 46 of FIG. 4. Whereas, if the subject 14 had little or no oxygenated hemoglobin in his/her skin (i.e. the subject is cold or dead) his/her base line, skin reflectance spectrum 3 would resemble a line sloping up to the left.

Nonetheless, once a base line, skin reflectance spectrum 34 (e.g. see FIG. 3) has been identified by the signal processor 28, comparisons between the base line, skin, reflectance spectrum 34 and other areas of the target's skin may be performed. Though only one other area need be examined. These other areas should be selected for their vulnerability to full participation in a blush.

For instance, in adults, the ears tend to blush relatively easily with the face and neck also susceptible to blushing. Additionally, because of the crenulated or ribbed structure of the ear, machine vision systems may require less processing to identify the ear than other blushing areas might require. Furthermore, the ears will likely contain about the same amount of melatonin as the face and hands. Accordingly, the image processor 28 may select the ears of the subject 14 for further scrutiny.

As previously noted a blush consists of an increase in dermal hydration and sub-dermal hemoglobin flow indicating that the subject 14 is experiencing physiological stress. The oxygenated hemoglobin tends to absorb incident radiation as shown by the oxygenated hemoglobin absorption spectrum 46 shown in FIG. 4. In particular, a low wavelength maximum 49 in the absorption spectrum causes a relatively large attenuation in a corresponding range of the skin reflectance during a blush. Local maxima 50 and 53 also cause similar attenuations in the ranges corresponding to the local maxima 50 and 53.

Thus, the typical blushing reflectance spectrum will include a low wavelength, low intensity range (corresponding to maximum 49) and a pair of mid wavelength, low reflectance ranges (corresponding to maxima 50 and 53). These attenuated areas of the blushing skin reflectance spectrum are caused by the increased hemoglobin absorbing radiation according to FIG. 4. Thus, it is the increased attenuation, over that of non blushing skin, exhibited at the attenuated regions (corresponding to maxima 49, 50, and 53) upon which the processor 28 bases the determination that a blush is present.

To aid in detecting the increased attenuation caused by a blush, a data base (not shown) may be accessed by the processor 28. From the data base the processor may extract normal, non blushing, and blushing skin reflectance data from one or more subjects similar in skin type to that of the subject 14. From the data, the processor may more precisely determine the range of wavelengths at which blush caused attenuation would occur and further characterize the amount of increased attenuation likely to be observed during a full blush. Accordingly, the system 10 may make a highly accurate determination of the presence of even a partial blush.

It should also be noted too that the palms have a more reddish tint than other areas of the body. Thus, per the present invention, if the image 22 contains an image of the palm, the palm spectrum can be used to verify that a blush has been positively identified. If the increased attenuation of the identified blushing spectrum resembles, or exceeds, the increased attenuation of the palm spectrum a high likelihood exists that the blush determination was successful. Note that the data base discussed above may also include data for the skin reflectance data for numerous, varied skin types thereby further enabling the system 10 to make a highly accurate blush determination.

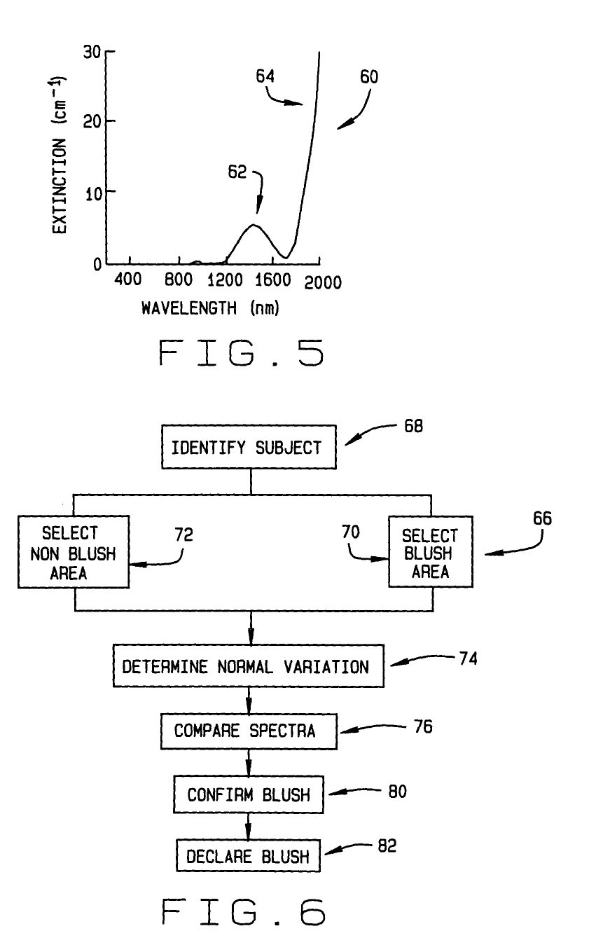

In addition, or in the alternative, the effect dermal hydration has on the reflected skin spectrum may be used to determine if a subject 14 is blushing. In particular, FIG. 5 shows a graph of the extinction coefficient of water. Since perspiration largely consists of water, the properties of perspiration will largely resemble the properties of water.

Also, of note, FIG. 5 shows the extinction coefficient of water 60 as opposed to a graph of the absorption coefficient (as shown for hemoglobin in FIG. 4). However, because the extinction coefficient is the sum of the absorption coefficient and the scattering coefficient, similar reasoning applies to the attenuation caused by hemoglobin and the attenuation caused by thermal hydration. It will be understood also that the extinction coefficient is usually given in terms of the fraction of light lost over a given distance.

Thus, as indicated in FIG. 5, dermal hydration will cause attenuation in the skin reflectance spectrum in ranges of high absorption 62 and 64. The effects, including those of a high derivative range associated with areas 62 and 64, may be used by the image processor 28 to determine or confirm that a blush has been identified.

One notable difference between the light absorption by hemoglobin and dermal hydration is that hemoglobin absorbs strongly in ranges of the visible spectrum. In contrast, perspiration is largely transparent to visible radiation. Instead perspiration absorbs strongly in ranges of the near infrared spectrum.

Accordingly, the presence of perspiration on the selected area of the subject 14 will cause attenuation of the skin reflectance spectrum 34 (shown in FIG. 3) in a range 62 near 1400 nanometer and especially in a range 64 above about 1700 nanometers. Thus, image processor 28 may determine, or confirm, the presence of a blush and stress by examining the intensity of the reflected spectrum for a selected area near 1400 nanometers and above 1700 nanometers for attenuation in a manner similar to that set forth herein with reference to hemoglobin. If dermal hydration is found, then the image processor 28 may determine or confirm that a blush is occurring.

At least one advantage of the present invention arises because of the examination of visible radiation for hemoglobin attenuation and infrared radiation for dermal hydration. Some unsophisticated subjects 14 may be aware enough of the possibility of surveillance to attempt masking their skin with material (e.g. makeup) effective in the visible spectrum yet totally ineffective in the infrared spectrum or visa versa. Thus, the present invention which may examine wavelengths in both ranges provides a mechanism to penetrate attempted camouflage.

In a preferred embodiment of the present invention, a method 66 may be seen depicted in FIG. 6. The method 66 consists of identifying a human subject 14 for subsequent examination in step 68. An area indicative of a blush and an area indicative of the subject's inherent skin type may then be selected in parallel, or series, in steps 70 and 72 respectively. Statistical comparisons of the spectrum from the two areas of skin may then be made to determine by how much one may vary from the other due to factors other than a blush as in step 74. Of course these statistical comparisons should be made outside of the ranges of interest (e.g. ranges near 542, 560, 576, 1400, and above 1700 nanometers) where large changes are to be expected due to blushing. Such a statistical comparison may suppress false alarms due to variations between the two areas of skin on the same subject. For instance, if the area subject to blushing happens to be in shade, the lessened intensity might otherwise be construed as blushed induced attenuation.

In step 76, the spectrum from the two areas may then be compared. In particular comparisons may be made near at least one of 542, 560, 576, 1400, and above 1700 nanometers to determine if a blush is present. If desired, in step 80, the result may be confirmed by more detailed analysis (e.g. high derivative areas 55, 56, and 57 may be examined) or the palm reflectance spectrum may be used. Based on the comparisons, if the blush susceptible area shows attenuation in one or more of the selected ranges a blush may be declared in step 82. If no attenuation, not enough attenuation, or attenuation in too few of the selected wavelengths is observed then a target is declared to be non-stressed.

As will be appreciated by those skilled in the art, the present invention provides a reliable system with which to detect physiological stress in human beings. Moreover, because of the image processing software according to the present invention, subtle changes in skin reflectance spectra indicative of an emerging blush may be detected before the human eye would notice the change. Additionally, methods to defeat camouflage or masking of a subject's skin have been presented.

While various preferred embodiments have been described, those skilled in the art will recognize modifications or variations which might be made without departing from the inventive concept. The examples illustrate the invention and are not intended to limit it. Therefore, the description and claims should be interpreted liberally with only such limitation as is necessary in view of the pertinent prior art.

1. A system for detecting physiological stress in a subject, the system comprising:
a processor adapted to receive an image of the subject from a camera, adapted to identify a first spectral characteristic of the subject when the subject is unstressed and adapted to identify a second spectral characteristic of the subject when stressed, and,
the processor further adapted to compare an area of the image with the first and the second spectral characteristics and adapted to indicate whether the subject is experiencing physiological stress based on which of the spectral characteristics the image more closely coincides with.

2. The system according to claim 1, the second characteristic further comprising being coincident with one of a spectrum of sub-dermal blood flow and a spectrum of dermal hydration, whereby the second characteristic indicates a blush.

3. The system according to claim 1, wherein the first and the second spectral characteristics differ at a frequency selected from the group consisting of about 542 nanometers, about 560 nanometers, about 576 nanometers, about 1400 nanometers, and about 1700 nanometers, and whereby the difference indicates a blush.

4. The system according to claim 1, the processor coupled to the camera.

5. The system according to claim 1, wherein the processor is coupled to an alarm and activates the alarm if the area of the image more closely coincides with the second spectral characteristic.

6. The system according to claim 5, wherein the processor is coupled to a time source, a date source, and a location source to enable the processor to associate the time, date, and location with the image.

7. The system according to claim 5, the wherein the system is installed in one of an airport, an interrogation room, and a store.

8. The system according to claim 1, wherein the processor identifies the first spectral characteristic from the image to detect an unstressed condition of the subject in real time.

9. The system according to claim 8, wherein the processor is adapted to identify the first spectral characteristic from a back of the hand of the subject.

10. The system according to claim 1, wherein the processor identifies the second spectral characteristic from the image to detect a stressed condition of the subject in real time.

11. The system according to claim 10, wherein the processor identifies the second spectral characteristic from a palm of the hand of the subject.

12. A method for detecting physiological stress of a subject, the method comprising:
observing an image of the subject with a system, the subject to include a first spectral characteristic when the subject is unstressed and a second spectral characteristic when the subject is stressed;
comparing an area of the image to the first spectral characteristic with the system;
comparing the area of the image to the second spectral characteristic with the system; and
determining with the system which of the spectral characteristics the area of the image more closely coincides with to detect if the subject is experiencing stress.

13. The method according to claim 12, further comprising selecting the second spectral characteristic from the group consisting of a spectrum of sub-dermal blood flow and a spectrum of dermal hydration and wherein the second spectral characteristic indicates a blush.

14. The method according to claim 12, wherein the comparisons further comprise comparing the image with the first and the second spectral characteristics near a frequency selected from the group consisting of about 542 nanometers, about 560 nanometers, about 576 nanometers, about 1400 nanometers, and about 1700 nanometers to determine a difference indicative of a blush of the subject.

15. The method according to claim 12, further comprising coupling a camera to the system whereby the camera inputs the image to the system.

16. The method according to claim 12, further comprising activating an alarm if the area of the image more closely coincides with the second spectral characteristic than the first spectral characteristic.

17. The method according to claim 16, further comprising associating a time, a date, and a location with the image.

18. The method according to claim 16, further comprising installing the system in one of an airport, an interrogation room, and a store.

19. The method according to claim 12, the method further comprising identifying the first spectral characteristic from the image in real time.

20. The method according to claim 19, the method further comprising identifying the first spectral characteristic from a back of a hand of the subject.

21. The method according to claim 12, further comprising identifying the second spectral characteristic from the image in real time.

22. The method according to claim 21, further comprising identifying the second spectral characteristic from a palm of a hand of the subject.

23. A system for detecting physiological stress in a subject, comprising:
a processor adapted to receive an image of a subject and adapted to identify a first and a second area of skin of the subject, the first area to be unlikely to blush, the second area to be likely to blush, and
the processor further adapted to compare the first and the second areas of skin and adapted to indicate whether the subject is experiencing physiological stress based on an attenuation at a pre selected frequency of a spectrum between the first and the second areas of skin.

24. The system according to claim 23, wherein the attenuation is representative of a change in one of a spectrum of sub-dermal blood flow and a spectrum of dermal hydration and wherein the attenuation indicates a blush.

25. The system according to claim 23, wherein the attenuation occurs near a frequency selected from the group consisting of about 542 nanometers, about 560 nanometers, about 576 nanometers, about 1400 nanometers, and about 1700 nanometers, whereby the difference indicates a blush.

26. The system according to claim 23, wherein the processor activates an alarm if the comparison indicates a blush.

27. The system according to claim 26, wherein the processor associates a time, a date, and a location of the subject with the image.

28. The system according to claim 26, wherein the system is installed in one of an airport, an interrogation room, and a store.
